(19) United States
(12) Patent Application Publication
(10) Pub. No.: US 2006/0057535 A1
(43) Pub. Date: Mar. 16, 2006

(54) Cordless intraoral dental examination instrument having non-plano mirror

(75) Inventor: Richard A. Tamburrino, Auburn, NY (US)

Correspondence Address: WALL MARJAMA & BILINSKI, 101 SOUTH SALINA STREET, SUITE 400, SYRACUSE, NY, 13202, US

(73) Assignee: Welch Allyn, Inc., Skaneateles Falls, NY (US)

(21) Appl. No.: 10/941,280

(22) Filed: Sep. 15, 2004

(51) Int. Cl. A61B 1/24; A61C 3/00

(52) U.S. Cl. 433/30; 433/29

(57) ABSTRACT

An intraoral dental mirror includes an elongated handle and a light guide that is releasably attached to a distal end of the handle. The light guide includes a mirrored portion that has a non-plano reflective surface that is configured to either magnify the image or to widen the field of view thereof as opposed to a flat or piano mirrored surface.

### FIELD OF THE INVENTION

This invention relates to the field of medical and dental diagnostic instruments, and more particularly to an illuminated dental intraoral instrument having a disposable mirror that includes an improved imaging surface.

### BACKGROUND OF THE INVENTION

Intraoral dental mirrors, such as those described for example, in U.S. Pat. No. 6,574,744B1 to Oshida et al., and U.S. Pat. No. 5,741,132 to Usui et al., are widely used in the field for examination of the teeth and other oral cavity structures. These mirrors typically include an elongated handle having a retroflexed reflective surface that is formed at a distal end of the handle. The handle portion of the instrument can accommodate at least one battery for powering a light source which is provided within the mirror portion of the instrument.

Other versions, such as those that are described in U.S. Pat. Nos. 5,139,421 and 5,457,611, each to Verberder et al., include a miniature light source, such as a halogen or other incandescent bulb or lamp, that is contained in a distal end of the elongated handle. A shank, interconnecting the mirror head with the elongated handle, includes a series of light pipes therewithin that transmit the light from the contained halogen bulb to the front and back of the mirror head. Alternatively, the light guide can be constructed from a light transmissive material that is releasably attached by means of a collet to the distal end of the elongated handle.

Though the above mirrored instrument is highly effective in examining the teeth and other oral structures of the patient, there is a general need in the field to improve the overall efficacy of examinations using the above instruments.

### SUMMARY OF THE INVENTION

It is therefore a primary object of the present invention to meet the above-noted general need in the field.

It is another primary object of the present invention to provide an improved intraoral dental mirror that provides improved examination capabilities for the dentist/hygienist.

Therefore and according to a preferred aspect of the present invention, there is provided an intraoral dental examination instrument comprising:

an elongated handle; and

a mirror releasably attached to a distal end of said handle, said mirror including a face portion having a non-plano reflective surface.

Preferably, the mirror is disposable and is releasably attached to the distal end of the handle of the examination instrument. The handle includes at least one contained battery as well as a miniature light source, such as at least one LED or halogen bulb, that is coupled optically to the end of the releasably attached mirror. The herein described mirror is made from a high quality, light transmissive material such as an acrylic, or other suitable material that promotes effective transmission from the light source to the face portion provided at the distal end of the mirror. The transmitted light is preferably dispersed from at least one light exit surface that is provided at a peripheral portion adjacent the face portion of the mirror, with the face portion being retroflexed relative to a primary axis of the mirror in order to permit enhanced viewing of the upper and lower arches of teeth.

Preferably and according to one aspect of the invention, at least one surface of the face portion is concave, thereby permitting magnification of the image as presented to the user. According to another aspect of the invention, the surface has a convex curvature in order to present a wider field of view to the user than that of typically known flat or plano mirrored surfaces. As such, the face surface of the herein described mirror can selectively be made to enhance examination of a patient in order to better observe the teeth and oral structures. For example, a number of adaptable mirrors can accompany the handle of the present instrument in order to provide this selectability with regard to a patient(s), as needed by the dentist or hygienist.

An advantage of the present invention is that the mirror is quickly detachable from the handle of the instrument and is disposable either for single-use or single patient use. The mirror is relatively inexpensive to manufacture and can be easily assembled and/or disassembled to and from the remainder of the instrument.

A further advantage of the present invention is that improved visibility is provided by use of a image magnifying or improved field of view features of the face surface of the releasably attachable mirror. Moreover, the mirror provides efficient and fairly direct illumination to the coupling of the LED or other miniature light source therewith. A further advantage of the present invention is that the herein described instrument can be used without need for overhead lighting. Additionally, the use of the instrument substantially eliminates the use of loupes or similar apparatus for inspection

These and other objects, features and advantages will become readily apparent from the following Detailed Description which should be read in conjunction with the accompanying drawings.

### BRIEF DESCRIPTION OF THE DRAWINGS

FIGS. 1(a) and 1(b) represents a side perspective view and a partially sectioned view, respectively, of a prior art intraoral dental examination instrument;

FIG. 2 is a top perspective view of an intraoral dental examination instrument made in accordance with a preferred embodiment of the present invention;

FIG. 3 is a sectioned view of the intraoral dental examination instrument of FIG. 2;

FIG. 4 is a side perspective view of the light guide of the intraoral dental examination instrument depicted in FIGS. 2 and 3;

FIG. 5 is a partial exploded view of the intraoral dental examination instrument of FIGS. 2 and 3;

FIG. 6 is an exploded view of an illumination cartridge used in the intraoral dental examination instrument of FIGS. 2-5;

FIG. 7 is a partial sectioned view of the illumination cartridge of FIG. 6 as assembled in the intraoral dental examination instrument of FIGS. 2-5;

FIGS. 8 and 9 are partial side sectioned views of the dental intraoral examination instrument of FIGS. 2-4 showing a retractable switch assembly illustrating same in respective OFF and ON positions;

FIG. 10 is a partial diagrammatic view of the optical coupling between the illumination cartridge of FIG. 6 and the releasably attached mirror in accordance with present invention;

FIG. 11 is an exploded view of a dental intraoral instrument made in accordance with another preferred embodiment of the present invention; and

FIG. 12 is an enlarged perspective view of a portion of the instrument of FIG. 11 depicting a mechanical detent used therewith.

### DETAILED DESCRIPTION

The following description relates to an intraoral dental examination instrument that is made in accordance with certain preferred embodiments of the present invention. Throughout the course of discussion that follows several terms, such as “top”, “side”, “lateral”, “bottom”, “distal”, “proximal”, “front”, “rear”, and the like are used in order to provide a convenient frame of reference with regard to the accompanying drawings. These descriptions, however, unless indicated otherwise, should not be regarded as limiting with regard to the present invention.

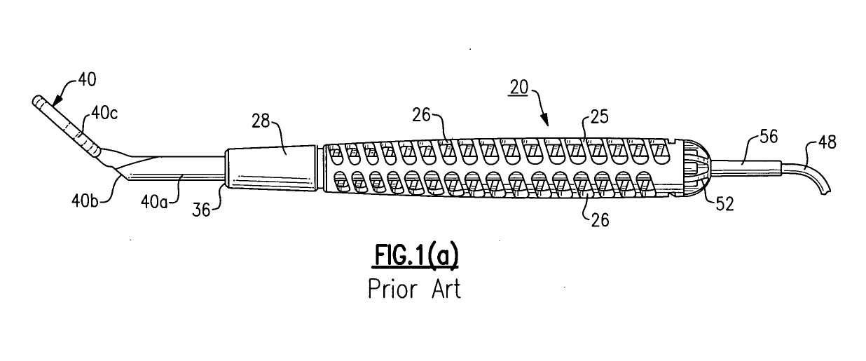

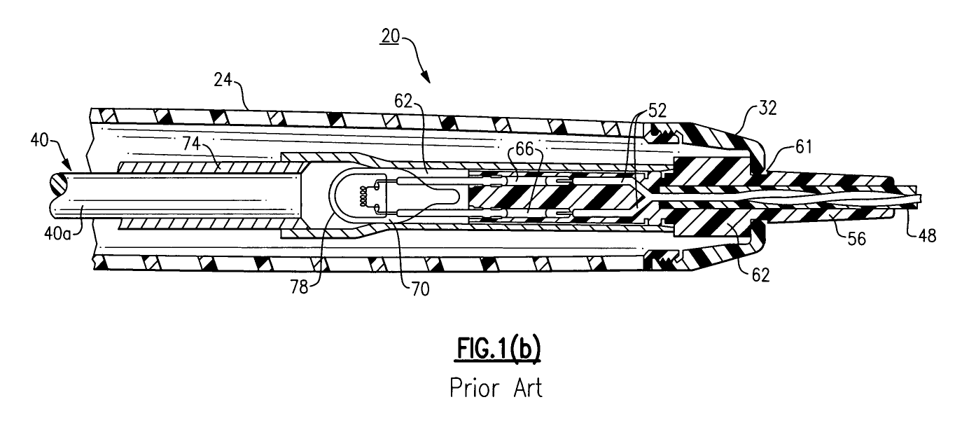

Prior to discussing the present invention and referring first to FIG. 1, there is illustrated an intraoral dental examination instrument 20 (partially shown according to the figure) that is made in accordance with the known art, described herein for background purposes. The examination instrument 20 is defined by an elongated body section 24, a front end cap 28 and a rear end cap 32, each preferably made from a moldable plastic or from any suitable low heat absorbing material.

Inserted into the front or distal end 36 of the front end cap 28 of the examination instrument 20 is a mirror 40 having a shank portion 40a, a heel portion 40b, and a face portion 40c. Mirror 40 is of the type that is described in greater detail in U.S. Pat. Nos. 5,457,611 and 5,139,421, in which light transmitted into the shank portion 40a from a contained bulb is emitted from the heel portion 40b and the face portion 40c on both facing sides of the mirror 40. The subject matter of each of the above-noted patents is herein incorporated by reference in its entirety. An electrical cord 48 containing a pair of wires 52 is inserted into the rear end cap 32, using a cord grip 56, to provide the energy from an exterior power supply (not shown) such as, for example, a 110 volt wall outlet, in order to operate the examination instrument 20.

The front end of the body section 24 of the instrument 20 includes a plurality of collet-like sections (not shown) as well as a threaded section (not shown) enabling interconnection of the front end cap 28 to the body section 24 as well as the mirror 40 to the instrument. When the mirror 40 is inserted into the front end of the body section 24, the front end cap 28 is rotated onto the end of the body 24 by virtue of mating threaded sections on each of the body and the front end cap, respectively, with the collet-like segments being caused to tighten around the shank portion 40a of the mirror 40, thereby holding the mirror securely in position within the elongated body section 24 of the instrument 20. Additional detail is provided in the above incorporated U.S. Pat. Nos. 5,457,611 and 5,139,421.

Referring to FIG. 1(b), the electrical cord 48 extending from an external power supply (not shown), is received within a molded lamp assembly unit 62 that is situated at the rear end of the body section 24 through an opening 61 formed in the rear end cap 32 and wires 52 are attached into the unit to a pair of electrical connectors 66. A miniature high intensity lamp 70, such as a miniature halogen bulb, is connected to the opposite ends of the connectors 66. The lamp 70 is positioned within the confines of the elongated body section 24 at one end of a heat sink 74 at close approximation to the shank portion 40a of the mirror 40, the latter being made from a light transmissive material, the lamp further containing a lens 78 that is configured and designed for focusing the light from the lamp into the end of the shank portion 40a. The heat sink 74 is extensive for purposes of this instrument 20 and includes separate portions that cover each of the lamp assembly unit 62 and light transmissive shank portion 40a of the mirror 40. In addition, a light weight gripping handle 25 covering the elongated body portion 24 includes a series of vent holes 26 also used for dissipating the heat that is generated by the instrument during use. As noted, additional details relating to this examination instrument 20 are described in U.S. Pat. No. 5,454,611, previously incorporated by reference herein.

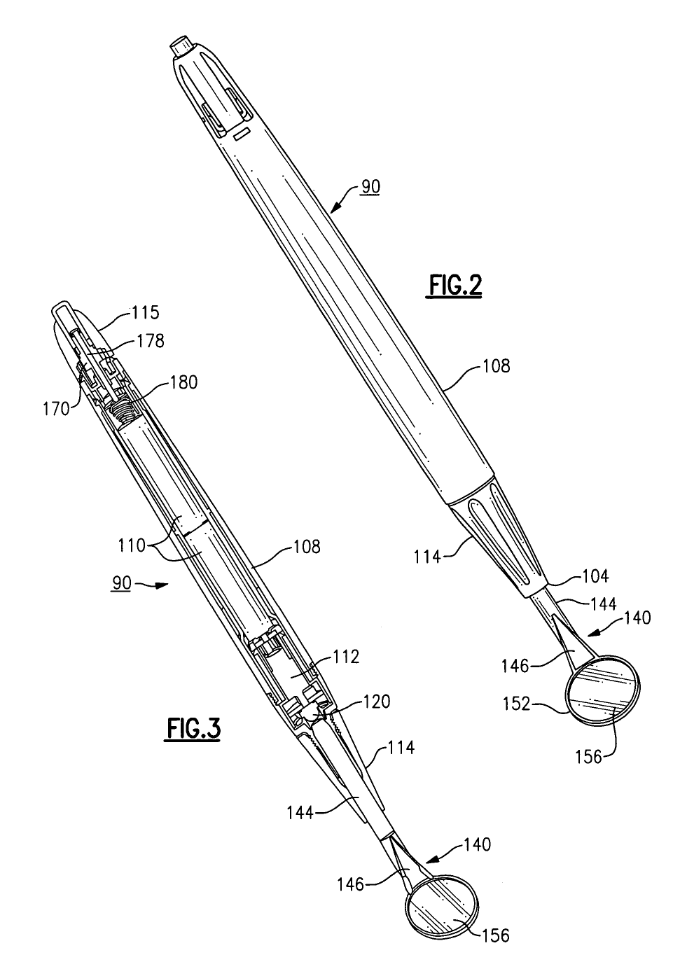

Referring now to FIGS. 2 and 3, there is shown an intraoral dental examination instrument that is made in accordance with a preferred embodiment of the present invention. The instrument 90, as now described in brief, includes an elongated body 108 that, like the preceding, includes a front end cap 114 and a rear end cap 115, respectively, each of which are releasably attached to the body as well as a mirror 100 that is preferably releasably attached to a distal end 104 of the instrument. The front end cap 114, includes threaded portions that engage with respective threaded portions formed in the front end of the body 108. The rear end cap 115, according to this embodiment snaps into place onto the rear or proximal end of the body 108. Preferably the body 108, as well as the front and rear end caps 114, 115, are each manufactured from plastic or a similar low heat absorbing material such as Radel-R, polyphenylsulfone or suitable polymer or other low heat absorbing material that permits autoclaving.

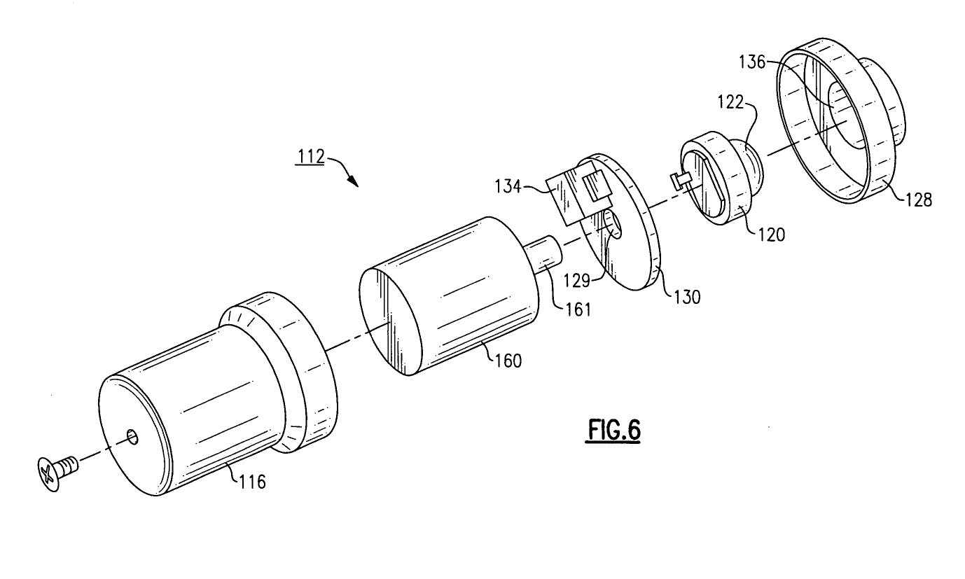

Unlike the preceding described examination instrument of FIGS. 1(a) and 1(b), however, the herein described examination instrument 90 does not require an exterior power supply, the present instrument being cordless and powered by a set of stacked batteries 110 that are retained within a defined battery compartment 111, FIG. 5, that is provided in the interior of the body 108. According to this embodiment, the batteries 110 are alkaline, but can also be nickel-cadmium or other suitable type, the batteries also preferably being rechargeable.

An illumination cartridge or housing 112 is also retained according to this embodiment within the body 108 adjacent the distal end 104 thereof and distal relative to the contained batteries 110, the cartridge being described in greater detail below. The batteries 110 are retained in biased relation within the body 108 of the instrument 90 relative to the illumination cartridge 112 by means of a compression spring 180 that is positioned adjacent the rear end cap 115.

The examination instrument 100 of this specific embodiment further includes a switch assembly 170, preferably contained in the rear or proximal end of the instrument 90, the switch assembly according to this preferred embodiment including a retractable plunger 178 containing a pin that moves between respective ON and OFF positions in order to selectively provide an electrical connection between the contained batteries 110 and the illumination cartridge 112 in order to energize an LED 120 retained by the cartridge, as will be described in greater detail below.

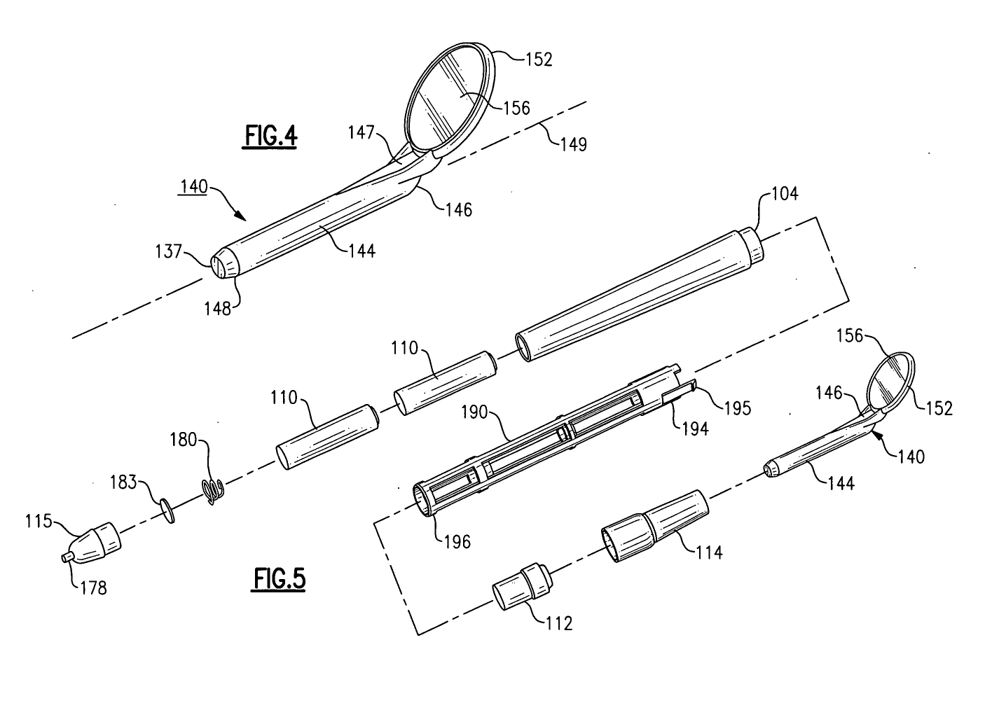

Prior to describing the interior features of the instrument 90, and referring first to FIGS. 2-4, the mirror 140 of the herein described instrument 90 includes an elongated shank portion 144. The shank portion 144 has a proximal end 148 that includes a defined light entrance surface 137, the shank portion terminating at a heel portion 146 having a pair of opposing substantially parallel light exit surfaces 147 on each of the top and bottom sides (only the top surface is shown in FIGS. 2-4) thereof, each of which are angled relative to a primary axis 149 that extends through the shank portion. A face portion 152 is integrally formed at the distal end of the mirror 140 along an axis (not shown) coextensive with that of the heel portion 146. The face portion 152 according to this embodiment is substantially circular in configuration (though other shapes could be contemplated) and includes a non-plano mirrored surface 156. The mirror 140 according to this embodiment is entirely made from a high-quality light transmissive material, such as acrylic, polyamide, polycarbonate and the like, and is preferably molded including the face portion 152 and the mirrored surface 156. The reflective portion of the mirror assembly 140 is molded according to this embodiment using “In Mold Decoration” technology wherein the contour of the mirrored surface 156 is controlled by the shape/contour of the mold. According to this embodiment, the non-plano mirrored surface 156 is concave in curvature in order to provide magnification of the object(s) being examined. According to this embodiment, about a 1.5× magnification is created although this parameter can easily be varied. It should be noted that in the alternative that the non-plano mirrored surface 156 can also be made with a convex curvature, wherein a minified image can be produced, thereby creating a wider field of view which can be useful for certain oral applications.

Referring to FIGS. 5-7, the illumination cartridge 112 of the described embodiment consists of a cylindrical housing body 116 made from Radel R, polyphenylsulfone or suitable polymer, but can be fabricated from literally any electrical insulating material, wherein a miniature LED 120 is disposed at a distal end 104 adjacent a reflector cap portion 128 that is attached in overlaying relation thereto. The illumination cartridge 112 further includes a printed circuit board (PCB) 130 that contains suitable electronics 134 thereupon used for powering the miniature LED 120. The PCB 130 according to this embodiment is disposed immediately below or proximal to the miniature LED 120, and includes an opening 129 for receiving the projecting portion 161 of a heat sink 160, retained by the housing body 116 in order to dissipate heat that is generated by the miniature LED, as well as the PCB 130.

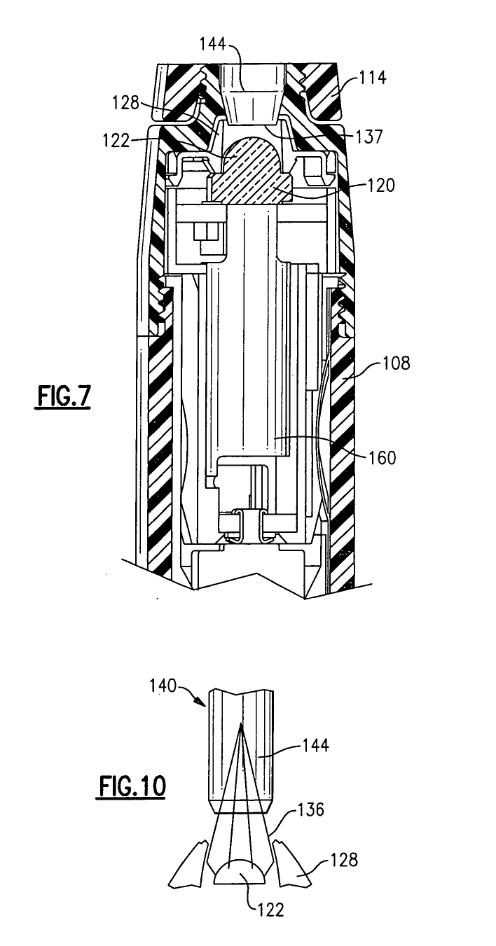

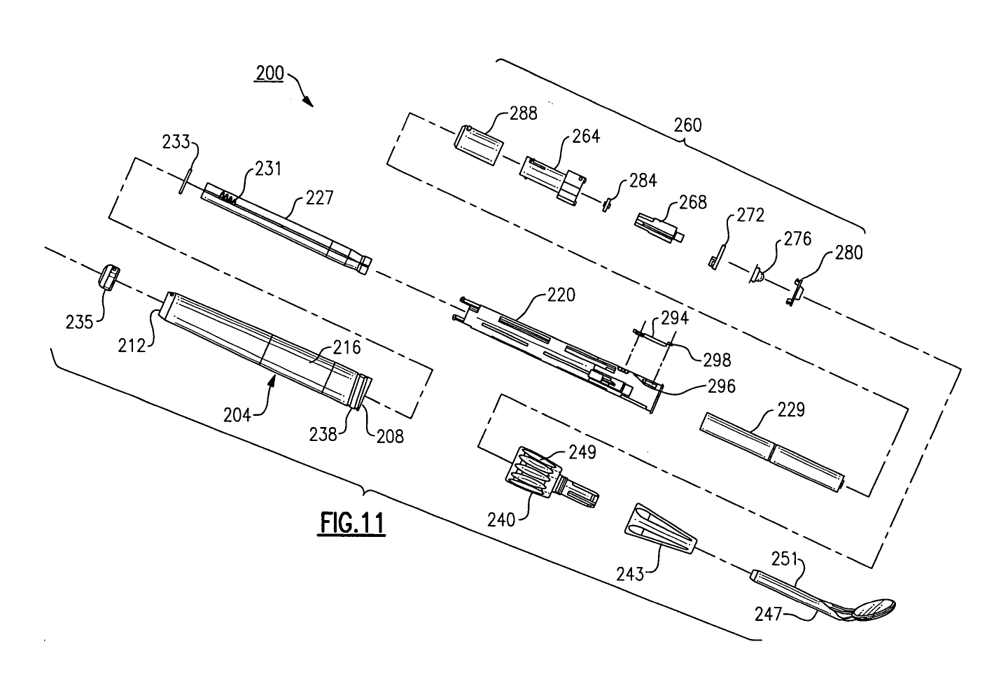

The reflector portion 128 is defined by a through opening 136 that is fitted about the lens envelope 122 of the miniature LED 120, the through opening preferably having an inwardly tapered surface 136 that is used in order to focus the light emitted from the miniature LED 120 onto a light entering surface 137 of the shank portion 144 of the mirror 140, as shown more particularly in FIGS. 7 and 10. The illumination cartridge 112 is retained by the front end cap 114 and is held in place by the body 108 and a contact strip 194 of an internal cage member 190. The reflector portion 128 also recesses the lens 122 and protects lens from damage, such as from dropping the cartridge 114, when removed from the instrument for cleaning.

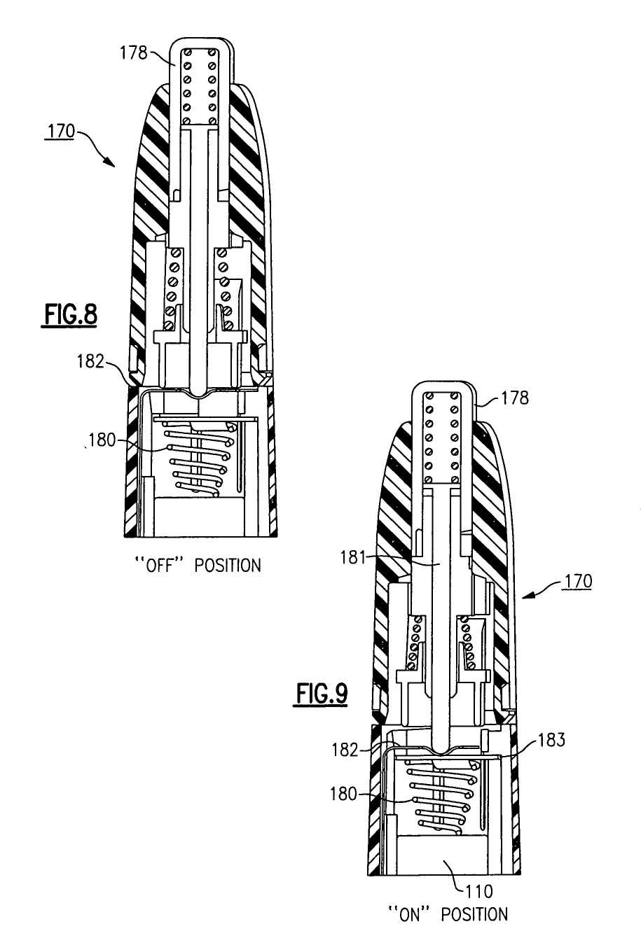

Referring to FIGS. 5, 8 and 9 and as previously noted herein, the examination instrument 90 is powered by means of a switch assembly 170 provided at the rear portion of the body 108. A retractable plunger 178 is mounted into the rear end cap 115 and includes a spring loaded pin member 181 that is selectively engageable with an S-shaped conformable spring member 182. As the retractable plunger 178 is pushed by the user from the OFF to the ON position, shown in FIGS. 8 and 9 respectively, the pin member 181 is caused to engage with the S-shaped spring member 182, which conforms and causes the battery contacts to engage with a conductive laterally extending contact strip 194 disposed on a hollow cylindrical internal cage member 190 covering the interior of the handle 108, the strip providing electrical connectivity with the contained cartridge 112, when the battery contacts engage the strip 194 as shown in FIG. 9, with the S-shaped spring member 182 in relation to electrical contacts. The internal cage 190 is used to hold and retain the conducting strip 194 and to provide centering for the batteries 110. The internal cage 190 further positions a small conductive plate 183 laterally, the plage being disposed adjacent to and proximal of the compression spring 180.

In operation, the mirror 100 is first attached to the examination instrument 90 using colleted portions (not shown) which are formed in the distal end of the handle 108. The front end cap 114 is then threaded onto the distal end of the handle 108 in order to secure the mirror 140 in place. The illumination cartridge 112 is attached to the distal end of the handle 108 by threaded portions. In this position, the shank portion 144 of the mirror 140 is situated in relation to the miniature LED 120.

The instrument 90 is initially in the “OFF” position shown in FIG. 8. As noted, depression of the retractable plunger 178 of the switch assembly 170 causes the contained pin member 181 to engage with the S-shaped spring member 182, causing relative movement of the S-shaped spring member and allowing the battery contacts to be brought into electrical contact with the illumination cartridge 112, by virtue of coextensive top and bottom ends 195, 196 respectively of the conductive laterally extending strip 194, thereby energizing the contained LED 120. Emitted light from the miniature LED 120 is then focused, by virtue of the reflector portion 128 onto the light transmissive shank portion 144 of the mirror 100. The mirror 140 thereby provides suitable illumination at the exit surfaces 147 of the heel portion 146 and the face portion 152, wherein the non-plano mirrored surface 156 permits enhanced examination of the teeth.

Following use, the mirror 140 can be removed by reversing the threading of the front end cap 114 and pulling same from the colleted areas of the body 108. The front end cap can then be removed, permitting access to the illumination cartridge 112 can be removed by reversing the threaded portion of the body 108 and the distal cap 114 from the body 108 along with the batteries 110, once the mirror 140 has also been removed from the distal end of the examination instrument 90. Once removed, the body 108 as well as the front and rear end caps 114, 115 can then be autoclaved.

Referring to FIG. 11, there is shown an intraoral dental examination instrument 200 made in accordance with a second preferred embodiment of the present invention. The examination instrument 200 according to this embodiment includes a plastic molded handle body 204, the body defined by a cylindrical configuration and having a pair of open ends 208, 212 that further define a hollow interior 216 which is sized for retaining a cylindrical interior cage member 220. The interior cage member 220 is preferably also made from plastic or similar insulating material which is also preferably autoclavable and includes a plurality of lateral slots that are defined along the axial length thereof, the interior cage member further having means for supporting an electrically conductive battery contact strip 227, the strip including a contact spring 231 at a proximal end thereof. The interior cage member 220 is also open-ended and has a hollow interior that defines a battery compartment which is appropriately sized for receiving a pair of stacked cadmium, alkaline or other suitable batteries 229. The contact spring 231 serves as a contact for the stacked batteries 229, relative to a battery contact disk 233 that is interposed between the contact spring and an endcap 235 that is fitted onto the proximal open end 212 of the handle body 204.

The distal end 208 of the handle body 204 of the examination instrument 200 includes a set of exterior screw threads 238 permitting the connection of a mirror support collet 240, as well as a collet locking ring 243 that retains a disposable mirror 247. The mirror support collet 240 is defined by a rotatable exterior knob 249 that includes a set of interior threads (not shown). The collet locking ring 243 is threadingly mounted onto the exterior of the mirror support collet 240, while the disposable mirror 247 includes an elongate shank portion 251 that is engaged with the collet locking ring, the collet locking ring being rotated to tighten or loosen the grip on the disposable mirror to permit engagement or disengagement therewith. The mirror 247 is similar in design to that previously described having the shank portion 251, a face portion and further including a non-plano reflective surface.

Disposed between the stacked batteries 229 and the mirror support collet 240 is an illumination assembly 260 that includes a hollow cylindrical cartridge body 264 containing a plurality of interconnected components. Among these contained components are a heat sink 268 onto which is packaged an LED driver circuit board 272. A miniature LED package 276 is disposed distally from the LED package 276 as well as a cartridge cap 280 having an inwardly tapered surface that is used to focus light from the miniature LED onto a light entering surface of the disposable mirror 247. A battery contact board 284 is disposed in the bottom of the cartridge body 264, each of which are held by a cartridge collar 288 that accommodates the illumination cartridge assembly 260 within the distal end of the handle body 204, when assembled thereto.

The herein described dental examination instrument 200 further utilizes a rotary switch assembly in order to selectively illuminate the miniature LED package 276 that is housed in the illumination cartridge assembly 260. The rotary switch assembly is enabled by rotating the rotatable exterior knob 249 of the mirror support collet 240 relative to the threads 238 of the distal end of the body 204, causing the illumination cartridge 260 to move into contact with the batteries 229. As the batteries 229 are pushed axially against spring 231 the strip 227 contacts cartridge collar 288 and creates a negative electrical contact. The battery contact 284 board contacts the positive battery terminals, completing the circuit and energizing the LED 276 with the strip 227 and contact disk 233

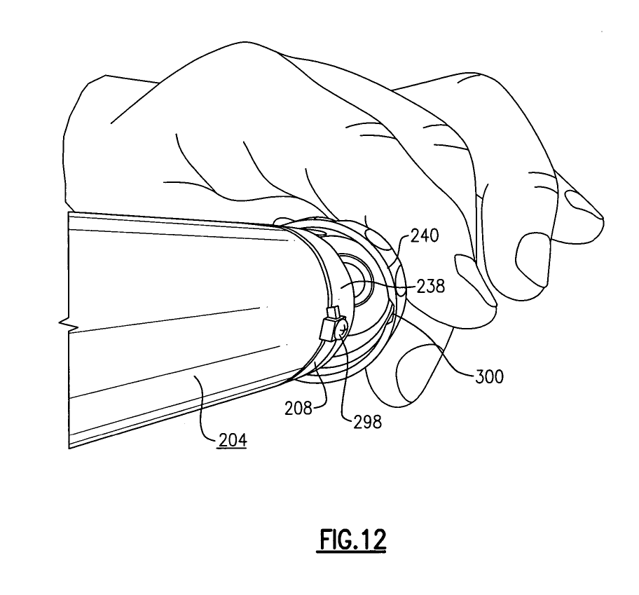

Referring to FIGS. 11 and 12 and in order for the user to properly sense actuation of the LED 276, a spring member 294 is preferably sandwiched between the exterior of the internal cage member 220 and the interior of the body 204. The spring member 294, in this instance, a leaf spring, is fastened at one end to a slot of the internal cage member adjacent the distal end thereof 220 by conventional means in this case, by means of an interference fit, the spring member including a depending engagement end portion 298 that initially resides in a groove 296 which is provided at the distal end of the internal cage member 220. When the internal cage member is inserted into the interior of the body 208, the spring member 294 is biased such that the engagement end portion 298 is caused to extend through a small opening (not shown) that is provided in the external threads 238 of the body 204 that is sized to accommodate same.

In operation, the mirror support collet 240 is attached to the distal end of the body 204 by threading the internal threaded portion thereof with the external threads 238 provided on the distal end 208 of the body. As the collet supporting ring 243 and collet 240 is rotated thereupon in a clockwise manner to initiate engagement, the LED 276 is caused to illuminate due to the above electrical connection that is created between the illumination cartridge 260, including the contained LED, and the batteries 229. As the above procedure is reversed, the LED 276 is deenergized upon counterclockwise rotation of the collet supporting ring 243 and collet 240 relative to the body 204 of the instrument whereupon the engagement end portion 298 is arranged relative to a hollow depression that is formed on the interior of the collet 240 into which the engagement end portion repeatably slips when the LED OFF position is reached as mechanically sensed by the user in the form of a detent.

### PARTS LIST FOR FIGS. 1-12

20 dental intraoral examination instrument
24 elongated body portion
25 gripping handle
26 slotted vent holes
28 front end cap
32 back end cap
36 distal end
40 mirror
40a shank portion
40b heel portion
40c face portion
48 electrical cord
52 wires
56 cord grip
61 opening
62 lamp assembly unit
66 electrical connectors
70 lamp
74 heat sink
78 lens
90 dental intraoral examination instrument
100 mirror
104 distal end
108 body
110 batteries
112 illumination cartridge
114 front end cap
115 rear end cap
116 housing body
120 LED
122 lens envelope
124 distal end
128 reflector portion
130 printed circuit board
131 opening
132 through opening
134 electrodes
136 inwardly tapered surface
137 light entering surface
140 mirror
144 shank portion
146 heel portion
147 light exit surface
148 proximal end
149 primary axis
152 face portion
156 non-plano mirrored surface
170 retractable switch assembly
178 plunger
180 spring, compression
181 pin member
182 spring member, S-shaped
183 conductive plate
190 internal case member
194 laterally extending electrical conductive strip
195 top end, strip
196 bottom end, strip
200 intraoral dental examination instrument
204 handle body
208 open end
212 open end
216 hollow interior
220 interior cage member
227 electrically conductive battery contact strip
229 batteries
231 contact spring
233 battery contact disk
235 endcap
240 mirror support collet
243 collet supporting ring
247 disposable mirror
251 elongate portion
260 illumination cartridge assembly
264 cartridge body
268 heat sink
272 LED driver circuit board
276 LED package
280 cartridge cap
284 battery contact board
288 cartridge collar
290 rotary switch assembly
294 spring
296 groove
298 engagement end portion
300 hollow depression

Other variations and modifications will be readily apparent to one of sufficient skill in the field that embody the inventive concepts taught in the above specification and are recited in the following claims.

We claim:

1. An intraoral dental instrument comprising:
an elongated handle; and
a mirror releasably attached to a distal end of said handle, said mirror including a face portion, said face portion having a non-plano reflective surface.

2. An intraoral dental instrument as recited in claim 1, wherein said light guide is disposable.

3. An intraoral dental instrument as recited in claim 1, wherein said handle includes a collet for releasably retaining said mirror.

4. An intraoral dental instrument as recited in claim 1, including at least one LED disposed in said handle, said at least one LED being disposed in relation to a proximal end of said mirror.

5. An intraoral dental instrument as recited in claim 4, wherein said non-plano reflective surface is concave in curvature in order to permit a mangified image.

6. An intraoral dental instrument as recited in claim 1, wherein said non-plano reflective surface portion is convex curvature to permit a wider field of view.

7. An intraoral dental instrument comprising:
an elongated handle; and
a mirror releasably attached to a distal end of said handle, said mirror including a face portion, said face portion having a surface with an enhanced field of view.

8. An intraoral dental instrument as recited in claim 7, wherein said surface of said face portion of said mirror is convex.

9. An intraoral dental instrument as recited in claim 7, wherein said mirror is disposable.
